(19) United States
(12) Patent Application Publication
(10) Pub. No.: US 2025/0017886 A1
(43) Pub. Date: Jan. 16, 2025

(54) FORMULATIONS AND METHODS FOR INCREASING CREATININE LEVELS IN PLASMA, EXTRACELLULAR FLUID AND/OR INTRACELLULAR COMPARTMENTS

(71) Applicant: Vireo Systems, Inc., Madison, TN (US)

(72) Inventors: Mark C. Faulkner, Madison, TN (US); Donald W. Miller, Winnipeg Manitoba (CA)

(73) Assignee: Vireo Systems, Inc., Madison, TN (US)

(21) Appl. No.: 18/764,786

(22) Filed: Jul. 5, 2024

Related U.S. Application Data
(60) Provisional application No. 63/525,264, filed on Jul. 6, 2023.

(51) Int. Cl. A61K 31/198; A61P 3/02

(52) U.S. Cl. CPC A61K 31/198; A61P 3/02

(57) ABSTRACT

Formulations and methods that increase creatinine levels in plasma, extracellular fluid and/or intracellular compartments in an animal and delivery methods therefor. In particular, the present disclosure is directed to creatinine pronutrients, formulations including the creatinine pronutrients, and methods of administering the formulations to increase creatinine levels in plasma, extracellular fluid and/or intracellular compartments.

### FIELD OF THE INVENTION

The present disclosure describes formulations and methods that increase extracellular and/or intracellular creatinine levels and delivery methods therefor. In this aspect, the present disclosure is directed to creatinine pronutrients, formulations including the creatinine pronutrients, and methods of administering the formulations to increase creatinine levels in plasma, extracellular fluid, and/or intracellular compartments, which, in turn, reduces or inhibits Bradykinin receptor activation, reduces or prevents loss of blood-brain barrier integrity, and/or reduces or inhibits activation of the inflammatory pathway.

### BACKGROUND OF THE INVENTION

Bradykinin (BK) is a pro-inflammatory nonapeptide that acts through it receptors and activates several second messenger systems to regulate blood-brain barrier permeability, pain perception, and nitric oxide production, among other things. Indeed, BK is released in pathological conditions such as trauma and inflammation and then binds to its kinin receptors B1 and B2. As a result, while expression of B1 is minimal under normal circumstances, it is expressed as a result of tissue injury and its signaling promotes local tissue inflammation by recruiting neutrophils, dilating capillaries, and constricting venous outflow.

In addition, Bradykinin receptors induce the overexpression of a number of cytokines, including TNF-α, IL-10, IL-6, IL-1β, IL-8 and IL-2 that are implicated in inflammation, respiratory, gastrointestinal, cardiac, neuronal, ophthalmologic and dermatological problems. It has been suggested that the BK receptor activation that occurs in neuroinflammatory diseases may contribute to both the inflammation and loss of blood-brain barrier integrity that is characteristic of these diseases (Mugisho O O, Robilliard L D, Nicholson L F B, Graham E S, O'Carroll S J. Bradykinin receptor-1 activation induces inflammation and increases the permeability of human brain microvascular endothelial cells. Cell Biol Int. 2020 January; 44 (1): 343-351). In sum, BK release leads to activation of B1 and B2 receptors, which, in turn, increases permeability of brain endothelial cells, and activates inflammatory pathways in the brain.

Creatine is an organic acid that is synthesized in the liver and kidneys from arginine, glycine, and methionine (Brosnan J T et al., Ann Rev Nutr 27:241-261 (2007); Greenhaff P, J Nutr Biochem 8:610-618 (1997); Wallimann et al., Amino Acids 40:1271-1296 (2011)). In the body, creatine is non-enzymatically converted into creatinine (Wyss M et al., Physiol Rev 80:1107-1213 (2000); Santos R V, et al. Life Sci 75:1917-1924 (2004)). The structure of creatinine is as follows:

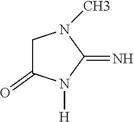

While creatinine has been considered an inert metabolic end product of creatine, studies have found that creatinine (and its derivatives such as creatinine HCl) possesses biological activity. Indeed, one study by Madan et al. (1979) indicates that creatinine acts as an anti-inflammatory agent against acute and chronic inflammation in rats. Another study by Leland et al. (Int Immunopharm 11:1341-1347 (2011)) suggested that creatinine may have the ability to dampen the innate immune response. In addition, a 2011 study demonstrated several downstream metabolites of creatinine and their reactive oxygen species scavenging effects (Drug Discoveries & Therapeutics 5 (4): 162-175 (2011)). In yet another study, the ability of creatinine and creatinine HCl to suppress bacterial replication was demonstrated (McDonald T L et al., J Antibiot (Tokyo) 65:153-156 (2012)). In a subsequent study, it was shown that creatinine HCl does not affect the growth of fungi, which was then used to demonstrate that creatinine HCl could be used as a novel additive in fungal growth media to permit fungi to grow efficiently without bacterial contamination (Smithee et al., J Microbiol Meth 105:155-161 (2014)). U.S. Pat. No. 11,058,655 describes the use of creatinine and creatinine salts as anti-inflammatory immunomodulators useful for treating an inflammatory condition or an autoimmune disease and/or inducing an immunomodulatory response.

Creatinine levels can vary depending on age, race, gender, and body mass. Blood creatinine levels of 50 μM to 100 μM is generally considered normal (Brosnan J T et al., Ann Rev Nutr 27:241-261 (2007); McDonald T L et al., J Antibiot (Tokyo) 65:153-156 (2012)). In fact, while fluctuation is normal, the average level for a healthy adult male should be between about 0.6 to 1.2 mg/deciliter, and for females, it should be between 0.5 to 1.1 mg/deciliter. Most in the medical community believe that low creatinine levels are relatively benign. Indeed, it is largely accepted that certain populations have below-normal blood creatinine levels, e.g., pregnant women, vegetarians, and those with low muscle mass due to age, chronic illness, and malnutrition, and no significant effort is made to re-balance those creatinine levels outside of treating the underlying condition (if applicable). While physical exercise may be recommended for the increase in muscle mass and a change in diet may be recommended to those who follow a low-protein diet, no further action is generally taken to adjust the blood creatinine levels. Moreover, it is recognized that it is difficult to improve creatinine levels in patients who are suffering from chronic illnesses and the measures that are usually taken similarly include an adapted diet and supportive care and an adapted diet.

In short, since low levels of creatinine are viewed as a normal part of aging or a temporary issue that can be resolved with changes to diet, there is no specific treatment for low creatinine levels or focus on increasing creatinine levels. This may be at least partially due to the fact that creatinine had historically been considered as an inert waste product with no active function and/or that higher than normal blood creatinine levels are associated with a reduced ability to properly filter and eliminate solutes from the blood—an important kidney function. Indeed, when blood creatinine levels are low, the kidney is likely functioning well and little focus is given to bringing those creatinine levels back to normal. However, given the potential beneficial properties of creatinine, it would be advantageous to have a way to normalize and/or increase creatinine levels in the body (blood/plasma and/or intracellularly and regardless of whether the body is incapable due to age, loss of muscle mass, or diet restrictions). Furthermore, since supplementing with creatinine is generally disfavored, a novel way of increasing blood/plasma and/or intracellular creatinine levels without direct supplementation with creatinine would be beneficial.

In addition, it would be advantageous to reduce or inhibit BK receptor activation and/or signaling and/or maintain normal levels of BK receptors. Moreover, it would be advantageous to reduce or inhibit BK-induced permeability increases, reduce or prevent loss of blood-brain barrier integrity, and/or reduce or prevent activation of the inflammatory pathway. The present disclosure provides formulations that do not contain creatinine, but rather creatinine pronutrient(s) that facilitate conversion into creatinine once in the body (either extracellularly or intracellularly) to provide for increased creatinine within the plasma, extracellular fluid and/or intracellular compartments, and reduce or inhibit BK receptor activation and/or signaling, reduce or inhibit BK-induced permeability increases, reduce or prevent loss of blood-brain barrier integrity, and/or reduce or prevent activation of the inflammatory pathway.

### SUMMARY OF THE INVENTION

The present disclosure is directed to a method for increasing creatinine levels in the body, the method including providing a formulation, the formulation comprising a therapeutically effective dose of at least one creatinine pronutrient. In one embodiment, the creatinine pronutrient includes ethyl (α-guanido-methyl) ethanoate, creatine alkyl ester, creatine amide alkyl ester, creatine cyclohexyl ester, and salts, derivatives, or combinations thereof. In another embodiment, the formulation includes about 30 to about 100 weight percent of the creatinine pronutrient.

The present disclosure also relates to a method for increasing extracellular creatinine levels in an animal, the method including: providing a formulation, the formulation including a therapeutically effective dose of at least one creatinine pronutrient; and administering a therapeutically effective dose of the formulation to the animal. In some embodiments, the creatinine pronutrient includes ethyl (α-guanido-methyl) ethanoate, creatine alkyl ester, creatine amide alkyl ester, creatine cyclohexyl ester, and salts, derivatives, or combinations thereof. In other embodiments, the creatinine pronutrient includes ethyl (α-guanido-methyl) ethanoate, creatine ethyl ester hydrochloride, creatine isopentyl ester hydrochloride, or derivatives or combinations thereof. In still other embodiments, the creatinine pronutrient includes creatine isopentyl ester hydrochloride or derivatives thereof. In this aspect, the formulation may include about 30 to about 100 weight percent of the creatinine pronutrient. In addition, the creatinine pronutrient may be present in the formulation at a concentration of about 0.1 mM to about 10 mM.

The present disclosure also relates to a method for increasing intracellular creatinine levels in an animal, the method including: providing a formulation, the formulation including at least one creatinine pronutrient; and administering a therapeutically effective dose of the formulation to the animal. In some embodiments, the creatinine pronutrient includes creatine tert-butyl ester hydrochloride or derivatives thereof. In other embodiments, the formulation includes about 30 to about 100 weight percent of the creatinine pronutrient. In still other embodiments, the creatinine pronutrient is present in the formulation at a concentration of about 0.1 mM to about 10 mM.

The present disclosure also relates to an oral supplement including a formulation including a creatinine pronutrient suitable for increasing creatinine levels in an animal, wherein the creatinine pronutrient includes creatine alkyl ester, creatine amide alkyl ester, creatine cyclohexyl ester or salts, derivatives, or combinations thereof, and wherein the creatinine pronutrient is present in the formulation at a concentration of about 0.1 mM to about 10 mM.

In some aspects, the formulation further includes a creatine derivative. In some embodiments, the creatinine pronutrient has a bioavailability that is at least 2 times the bioavailability of creatinine. In other embodiments, the creatinine pronutrient includes ethyl (α-guanido-methyl) ethanoate, creatine ethyl ester hydrochloride, creatine isopentyl ester hydrochloride, creatine tert-butyl ester hydrochloride, or derivatives or combinations thereof. In still other embodiments, the creatinine pronutrient includes creatine isopentyl ester hydrochloride or derivatives thereof. In yet other embodiments, the creatinine pronutrient includes creatine tert-butyl ester hydrochloride or derivatives thereof.

The present disclosure also relates to one or more creatinine pronutrients or a composition including one or more creatinine pronutrients that increase levels of creatinine in the body to reduce or inhibit BK receptor activation and/or signaling, reduce or inhibit permeability of BK across the blood-brain barrier, and/or reduce or prevent activation of the inflammatory pathway.

The present disclosure also relates to a method for reducing permeability increases across the blood-brain barrier in an animal due to bradykinin release, the method including: providing a formulation, the formulation including at least one creatinine pronutrient at a concentration of about 0.1 mM to about 10 mM; and administering a therapeutically effective dose of the formulation to the animal. In some embodiments, the creatinine pronutrient includes ethyl (α-guanido-methyl) ethanoate, creatine ethyl ester hydrochloride, creatine isopentyl ester hydrochloride, creatine tert-butyl ester hydrochloride, or derivatives or combinations thereof. In other embodiments, the creatinine pronutrient includes creatine isopentyl ester hydrochloride or derivatives thereof. In still other embodiments, the creatinine pronutrient includes creatine tert-butyl ester hydrochloride or derivatives thereof.

### BRIEF DESCRIPTION OF THE DRAWINGS

Further features and advantages of the invention can be ascertained from the following detailed description that is provided in connection with the drawings described below:

FIGS. 1A-1B are graphical illustrations of creatinine intracellular levels after dosing with a creatinine pronutrient according to an embodiment of the present disclosure:

FIGS. 2A-2C are graphical illustrations of plasma creatine levels after dosing with creatine monohydrate;

FIGS. 3A-3C are graphical illustrations of creatinine pronutrient levels after dosing with a creatinine pronutrient according to an embodiment of the present disclosure;

FIGS. 4A-4C are graphical illustrations of creatinine levels after dosing with a creatinine pronutrient according to another embodiment of the present disclosure;

FIG. 5 is a graphical illustration of permeability increases with Bradykinin and inhibition thereof with creatinine pronutrient according to an embodiment of the present disclosure;

FIG. 6 is a graphical illustration of permeability increases in cancer patient and inhibition thereof with creatinine pronutrient in according to an embodiment of the present disclosure;

FIG. 7 is a graphical illustration of prostaglandin release when exposed to inflammatory stimuli and inhibition thereof with creatinine pronutrient according to an embodiment of the present disclosure;

FIG. 8 is a graphical illustration of the lack of inhibition of COX activity with creatinine pronutrient according to an embodiment of the present disclosure; and

FIG. 9 is a graphical illustration of the effect of a creatinine pronutrient according to an embodiment of the present disclosure in a Bradykinin skin prick test.

### DETAILED DESCRIPTION OF THE INVENTION

The present disclosure is directed to creatinine pronutrients, formulations including such creatinine pronutrients, and methods of administering such pronutrients and/or formulations to raise creatinine levels in an animal. In some aspects, the formulations include one or more creatinine pronutrients that increase the amount of creatinine in extracellular fluid or plasma compartments. In other aspects, the formulations include one or more creatinine pronutrients that increase the amount of intracellular creatinine. In still other aspects, the formulations include one or more creatinine pronutrients that increase the amount of creatinine extracellularly and intracellularly.

Without being bound by any particular theory, it is contemplated that an increase in extracellular creatinine levels after exposure to or administration of the formulations of the present disclosure stem from non-enzymatic degradation of the one or more creatinine pronutrients in the formulation to creatinine. Likewise, without being bound by any particular theory, it is contemplated that an increase in intracellular creatinine levels after exposure to or administration of the formulations of the present disclosure stem from either (a) the non-enzymatic degradation of the creatinine pronutrient to creatinine in the extracellular fluid, which then enters the cells or (b) the creatinine pronutrient efficiently entering the cells and then converting to creatinine within the various intracellular compartments.

Moreover, it is contemplated that the formulations disclosed herein, when administered directly to a patient, optimize creatinine levels in the body through increased bioavailability. Without being bound by any particular theory, it is believed that the increased bioavailability may be the result of increased permeability across the GI tract and/or increased solubility of the creatinine pronutrient. In fact, increased creatinine levels may be achieved in patients that have lower than normal blood creatinine levels due to age, diet, muscle-wasting disease, chronic illness, or a combination thereof through administration of the creatinine pronutrients and formulations of the present disclosure.

The creatinine pronutrients and formulations of the present disclosure and administration thereof directly or indirectly effect the inflammatory response. For example, the pronutrients and formulations of the present disclosure and administration thereof reduce or inhibit BK receptor activation and/or signaling. In addition, the pronutrients and formulations of the present disclosure and administration thereof reduce or inhibit BK-induced permeability increases in brain endothelial cells. Without being bound by any particular theory, since leaky brain capillary endothelial cells are believed to be linked to an increase in prostaglandin release, which, in turn, triggers the inflammatory responses in the brain (central) and peripheral tissues, a reduction or inhibition of BK-induced permeability increases in brain endothelial cells is believed to also reduce inflammation both centrally and peripherally.

### Creatinine Pronutrient

A creatinine pronutrient suitable for use in accordance with the present disclosure has the ability, when included in a formulation made in accordance with the present disclosure, to increase creatinine levels in the plasma and extracellular fluid compartments through non-enzymatic degradation of the creatinine pronutrient to creatinine. In addition, a creatinine pronutrient suitable for use in accordance with the present disclosure has the ability, when included in a formulation made in accordance with the present disclosure, to increase intracellular creatinine levels through the non-enzymatic degradation of the creatinine pronutrient to creatinine in the extracellular fluid thus allowing the creatinine to enter the cells and/or by facilitating increased entry of the creatinine pronutrient into the cells and subsequent conversion to creatinine within the various intracellular compartments.

In some embodiments, the permeability, i.e., the ability of the compound to penetrate across a barrier, such as a membrane, cell wall, and the like, of the creatinine pronutrients disclosed herein may be greater than creatinine. In particular, without being bound by any particular theory, the formulations of the present disclosure may possess the ability to increase intracellular creatinine levels due to enhanced permeability properties of the creatinine pronutrient compared to creatinine. In this aspect, the permeability of the creatinine pronutrient may be at least about 30 percent greater than the permeability of creatinine across Caco-2 monolayers (used as an in vitro model for intestinal absorption). In other embodiments, the permeability of the creatinine pronutrient may be at least about 40 percent greater than the permeability of creatinine across Caco-2 monolayers. In still other embodiments, the permeability of the creatinine pronutrient may be at least about 50 percent greater than the permeability of creatinine across Caco-2 monolayers. In yet other embodiments, the permeability of the creatinine pronutrient may be at least about 60 percent greater than the permeability of creatinine across Caco-2 monolayers.

Furthermore, permeability studies conducted in Caco-2 monolayers demonstrate that the creatinine pronutrients disclosed herein have a higher permeability coefficient than creatine monohydrate and/or creatine citrate. In particular, creatine monohydrate has an apparent permeability coefficient of about 0.12±0.004 10-6 cm/s (see, e.g., Gufford, B., et al. pH-Dependent Stability of Creatine Ethyl Ester: Relevance to Oral Absorption. J Diet Suppl. 10 (3): 241-251 (September 2013)). In addition, permeability studies conducted in Caco-2 monolayers demonstrate that creatine citrate at a concentration of 10, 100, and 1000 μM in the donor compartment have a permeability coefficient of about 1.00×10⁻⁵, about 9.00×10⁻⁶, and about 6.00×10⁻⁶, respectively. In comparison, the creatinine pronutrients have a permeability coefficient of at least 0.6×10⁻⁶ cm/s when assessed in Caco-2 monolayers. For example, in some embodiments, the permeability coefficient of a creatinine pronutrient disclosed herein is greater than about 1.2×10⁻⁶ cm/s when assessed in Caco-2 monolayers. In other embodiments, the creatinine pronutrient has a permeability coefficient of greater than about 1.8×10⁻⁶ cm/s when assessed in Caco-2 monolayers. In still other embodiments, the permeability coefficient of the creatinine pronutrient is up to about 2.0×10⁻⁶ cm/s. In yet other embodiments, the permeability coefficient of the creatinine pronutrient is up to about 5.0×10⁻⁶ cm/s, up to about 1×10⁻⁵ cm/s, up to about 5×10⁻⁵ cm/s.

Moreover, the creatinine pronutrients as disclosed herein have improved cell penetration over that of creatine monohydrate and creatine citrate salt. Without being bound by any particular theory, the creatinine pronutrients have more lipophilicity for better tissue and cell penetration (as compared to creatine monohydrate and creatine citrate salt. More specifically, it is contemplated that laboratory analysis performed in Madin Darby canine kidney (MDCK) monolayers show that flux per hour with a creatinine pronutrient is at least double that of creatine monohydrate. In other words, if 10% of the original amount of creatine monohydrate added to one side of the MDCK monolayer made it across to the other side in a 60-minute period, at least about 20% of the original amount of the creatinine pronutrient added to one side of the MDCK monolayer will make it across to the other side over the same time period.

In other embodiments, the creatinine pronutrient may have a high solubility (as compared to other forms of creatine such as creatine monohydrate and creatine citrate) and a solubility that is greater than creatinine (23 mg/ml). In this aspect, the solubility, i.e., the amount of the compound that may be dissolved, of the pronutrient is at least about 100 mg/ml at room temperature (25° C.) after a time period of about 1.5 hours. In fact, the solubility of the pronutrient is greater than that of creatine monohydrate and creatine citrate salt. In some embodiments, the pronutrient has a solubility of about 120 mg/ml or greater at room temperature (25° C.) after a time period of about 1.5 hours. In other embodiments, the pronutrient has a solubility of at least about 150 mg/ml at room temperature (25° C.) after a time period of about 1.5 hours. In still other embodiments, the pronutrient has a solubility of about 175 mg/ml or greater at room temperature (25° C.) after a time period of about 1.5 hours. In yet other embodiments, the solubility of the pronutrient is about 200 mg/ml at room temperature (25° C.) after a time period of about 1.5 hours. In some embodiments, the solubility of the creatinine pronutrient is about 150 mg/ml to about 650 mg/ml at 25° C. for a time period of about 1.5 hours. In particular, the creatinine pronutrient may have a solubility of more than about 500 mg/ml, more than about 550 mg/ml, and more than about 600 mg/ml at 25° C. for a time period of about 1.5 hours. In another embodiment, the aqueous solubility of the compounds ranges from about 100 mg/ml to about 700 mg/ml at 25° after a time period of about 1.5 hours. In yet another embodiment, the compounds have an aqueous solubility of about 150 mg/ml to about 650 mg/ml at 25° after a time period of about 1.5 hours.

In addition, a creatinine pronutrient suitable for use in accordance with the present disclosure has the ability, when included in a formulation made in accordance with the present disclosure, to increase creatinine metabolites. In this aspect, since certain creatinine metabolites have been associated with scavenging free radicals and/or a reduced inflammatory response, a formulation including a creatinine pronutrient may have beneficial effects associated with reducing the inflammatory response and/or scavenging free radicals. Examples of such creatinine metabolites include, but are not limited to 5-hydroxy-1-methyl hydantoin (HMH). Without being bound by any particular theory, an increase in HMH may have beneficial anti-inflammatory effects due to reducing or inhibiting BK receptor activation and/or signaling and/or reducing or preventing loss of integrity of the blood-brain barrier, which in turn, reduces or inhibits the inflammatory response brought about by an increase in BK.

Suitable creatinine pronutrients include ethyl (α-guanido-methyl) ethanoate, creatine alkyl esters, creatine cycloalkyl esters, creatine amide alkyl esters, derivatives and salts thereof, and combinations thereof. Ethyl (α-guanido-methyl) ethanoate may be produced via acid-catalyzed conjugation of three amino acids. Once conjugated, ethyl (α-guanido-methyl) ethanoate has the following formula:

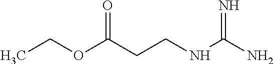

Examples of suitable creatine alkyl esters include, but are not limited to, creatine ethyl ester, which has a formula as follows:

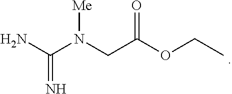

creatine isopentyl ester, which has a formula as follows:

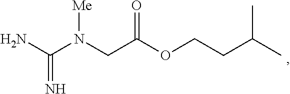

and combinations thereof.

Creatine ethyl ester may be produced as described in U.S. Pat. Nos. 10,531,680 and 6,897,334, the entire disclosures of which are incorporated by reference herein. Examples of creatine ethyl ester derivatives suitable for use as the creatinine pronutrient in accordance with the present disclosure include, but are not limited to, creatine phosphoester, mono-creatine glycerol, di-creatine glycerol, tricreatine glycerol, and combinations thereof.

Salts of creatine alkyl ester are also suitable for use as the creatinine pronutrients. For example, the creatinine pronutrient may be a creatine isopentyl ester as shown below:

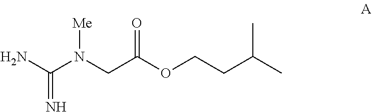

where A is a salt. In some embodiments, A is chloride, bromide, sulfate, or phosphate. In other embodiments, A is hydrogen chloride, hydrogen bromide, hydrogen sulfate, or hydrogen phosphate. For example, the creatinine pronutrient may be creatine ethyl ester hydrochloride, creatine tert-butyl ester hydrochloride, creatine isopentyl ester hydrochloride, and combinations thereof. In still other embodiments, A is mesylate or tosylate. In yet other embodiments, A is succinate, tartrate, or acetate.

A nonlimiting example of a suitable creatine cycloalkyl ester includes creatine cyclohexyl ester, which has a formula as follows:

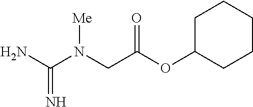 [SMILES: CN(CC(=O)OC1CCCCC1)C(=N)N]

Salts of creatine cycloalkyl ester are also suitable for use as the creatinine pronutrients. For example, the creatinine pronutrient may be a creatine cyclohexyl ester salt as shown below:

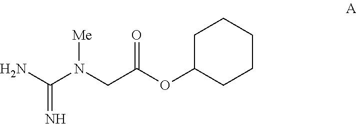

where A is a salt. In some embodiments, A is chloride, bromide, sulfate, or phosphate. In other embodiments, A is hydrogen chloride, hydrogen bromide, hydrogen sulfate, or hydrogen phosphate. In still other embodiments, A is mesylate or tosylate. In yet other embodiments, A is succinate, tartrate, or acetate.

Examples of suitable creatine amide alkyl esters include those having the following formula:

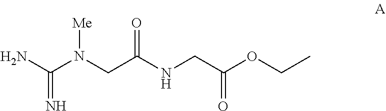

where A is a salt. In some embodiments, A is chloride, bromide, sulfate, or phosphate. In other embodiments, A is hydrogen chloride, hydrogen bromide, hydrogen sulfate, or hydrogen phosphate. For example, the creatinine pronutrient may be creatine glycinamide ethyl ester. In still other embodiments, A is mesylate or tosylate. In yet other embodiments, A is succinate, tartrate, or acetate.

### The Formulation

A formulation made in accordance with the present disclosure includes an effective amount of at least one creatinine pronutrient disclosed herein. For example, in one embodiment, the formulation includes an effective amount of creatine alkyl ester. In another embodiment, the formulation includes an effective amount of ethyl (α-guanido-methyl) ethanoate. As used herein, the term “effective amount” refers to an amount of the compound necessary or sufficient to elicit the desired biological or medical response, including the amount of a compound that, when administered to a subject for treating a disease or condition, is sufficient to affect such treatment for the disease. More specifically with respect to the creatinine pronutrient, “effective amount” refers to the amount of creatinine pronutrient necessary or sufficient to elicit the increase intracellular creatinine levels or optimization of blood creatinine levels. The effective amount will vary depending on the particular creatinine pronutrient, and characteristics of the subject to be treated, such as age, weight, etc. The effective amount can include a range of amounts. As is understood in the art, an effective amount may be in one or more doses, i.e., a single dose or multiple doses may be required to achieve the desired treatment endpoint. An effective amount may be considered in the context of administering one or more compounds, and a single compound may be considered to be given in an effective amount if, in conjunction with one or more other agents, a desirable or beneficial result may be or is achieved.

Determination of an effective amount is well within the capability of those skilled in the art, especially in light of the detailed disclosure provided herein. For any preparation used in the disclosed methods, the effective amount or dose can be estimated initially from in vitro and cell culture assays. For example, a dose can be formulated in animal models to achieve a desired circulating concentration or titer of the creatinine pronutrient. Such information can be used to more accurately determine useful doses in humans through allometric scaling.

In one embodiment, an effective amount of the creatinine pronutrient ranges from about 30 percent by weight to about 100 percent by weight of the formulation. In another embodiment, an effective amount of the creatinine pronutrient ranges from about 50 percent by weight to about 100 percent by weight of the formulation. In yet another embodiment, an effective amount of the creatinine pronutrient ranges from about 60 percent by weight to about 100 percent by weight of the formulation. In still another embodiment, an effective amount of the creatinine pronutrient ranges from about 70 percent by weight to about 100 percent by weight of the formulation. In another embodiment, an effective amount of the creatinine pronutrient ranges from about 80 percent by weight to about 100 percent by weight of the composition.

In this aspect, the formulation may include an effective amount of at least one creatinine pronutrient of about 200 mg/g to about 2000 mg/g per dose. In another embodiment, the effective amount of at least one creatinine pronutrient ranges from about 300 mg/g to about 1800 mg/g per dose. In still another embodiment, the effective amount of at least one creatinine pronutrient ranges from about 500 mg/g to about 1500 mg/g per dose.

An effective dose may include about 500 mg to about 1500 mg per 100 pounds body weight of the creatinine pronutrient. For example, in one embodiment, the effective dose may be from about 1500 mg to about 3000 mg of the creatinine pronutrient for a subject that weighs up to 250 pounds. In one embodiment, the effective dose of the creatinine pronutrient is from about 2250 mg to about 4500 mg for a subject that weighs over 250 pounds. In another embodiment, the effective dose of the creatinine pronutrient is from about 750 mg to about 1500 mg per 100 pounds body weight. Depending on the method of administration (discussed in greater detail below), blood creatinine level, and/or components of the formulation, the effective dose may be administered once per day or more than one time per day. In one embodiment, the effective dose of the creatinine pronutrient is between about 2 to about 5 grams, daily.

In some embodiments, the formulation includes a combination of at least two creatinine pronutrients. Without being bound by any particular theory, combinations of creatinine pronutrients may provide additive or synergistic effects. In this aspect, any of the creatinine pronutrients described herein may be combined in a formulation. The relative amount of a first creatinine pronutrient to a second creatinine pronutrient may vary depending on the desired effect. For example, the first to second creatinine pronutrient ratio may be 90:10 to 10:90. In some embodiment, the ratio of first creatinine pronutrient to second creatinine pronutrient may be 80:20 to 20:80.

Similarly, the combination of a creatinine pronutrient and a second creatine derivative or additive may have additive or synergistic effects. As such, in other embodiments, In this regard, the second creatine derivative may be creatine hydrochloride, creatine glycerol laurate ester, creatine propylene glycol laurate ester, creatine amide tert butyl ester, dicreatine glycerol ester, tricreatine glycerol ester, betaine cyclopentyl ester, creatine mesylate, creatine citrate, creatine pyruvate, or combinations thereof.

In other embodiments, the formulation includes a combination of at least one creatinine pronutrient and a second creatine derivative or additive. In this regard, the second creatine derivative may be creatine hydrochloride, creatine glycerol laurate ester, creatine propylene glycol laurate ester, creatine amide tert butyl ester, dicreatine glycerol ester, tricreatine glycerol ester, betaine cyclopentyl ester, creatine mesylate, creatine citrate, creatine pyruvate, or combinations thereof.

In this aspect, a formulation may include about 50 percent to about 95 percent by weight of the creatinine pronutrient and about 5 percent to about 50 percent by weight of the second creatine derivative. In some embodiments, the formulation may include about 60 percent to about 90 percent by weight of the creatinine pronutrient and about 10 percent to about 40 percent by weight of the second creatine derivative. In other embodiments, the formulation may include about 75 percent to about 95 percent by weight of the creatinine pronutrient and about 5 percent to about 25 percent by weight of the second creatine derivative.

Moreover, the addition of other components may further enhance such compositions whether they include one or more of the antiviral therapeutic or cellular energy compounds of the invention. For example, proteins, peptides, amino acids, simple and complex carbohydrates, lipids, fats, fibers, or combinations thereof may be included in the formulations discussed herein. Suitable proteins include serum albumin such as human serum albumin (HSA), recombinant human albumin (rHA), gelatin, casein, and combinations thereof. Suitable amino acids include, but are not limited to alanine, glycine, arginine, betaine, histidine, glutamic acid, aspartic acid, cysteine, lysine, leucine, isoleucine, valine, methionine, phenylalanine, aspartame, and combinations thereof. Suitable carbohydrates include, but are not limited to monosaccharides such as fructose, maltose, galactose, glucose, D-mannose, D-ribose, sorbose, and combinations thereof; disaccharides, such as lactose, sucrose, trehalose, cellobiose, and combinations thereof; polysaccharides, such as raffinose, melezitose, maltodextrins, dextrans, starches, and combinations thereof; and alditols, such as mannitol, xylitol, maltitol, lactitol, xylitol, sorbitol (glucitol), myoinositol, and combinations thereof.

The formulation may also include a homeopathic compound, a co-medication, a nutraceutical, a plant extract, a herbal preparation, a cosmetic agent, a pharmaceutical, or combinations thereof. In another embodiment, the formulation includes at least two of these other compounds. Suitable homeopathic compounds include, but are not limited to, actaca spicata, aesculus hippocastanum, arnica montana, belladonna, bellis perennis, bryonia, calcarea carbonica, calcarea fluorica, calc sulph MM, causticum, cayenne, cimicifuga racemosa, formicum acidum, hamamelis virginiana, hypericum perforatum, magnesia phosphorica, phytolacca decandra, pulsatilla, rhododendron chrysanthum, rhus toxicodendron, ruta graveolens, salicylicum acidum, sepia, sulphu, turmeric, green tea extract, grape extract, foeniculum vulgare, bellis perrinis, boswellia serrate, bromeliacaea, devil's claw (harpagophytum procumbens), bromelain, cordyalis yanhusuo, or combinations thereof. In addition, the formulations may include vitamins such as vitamins C, D, and E.

The formulations may further include at least one of any suitable auxiliaries including, but not limited to, diluents, binders, stabilizers, buffers, salts, lipophilic solvents, preservatives, adjuvants, or the like. For example, in some embodiments, the formulation may include a surfactant, alone or in combination with other surfactants, to stabilize the formulation. The surfactant can be selected from a variety of known anionic surfactants, cationic surfactants, zwitterionic surfactants, nonionic surfactants, surface active biological modifiers, and combinations thereof. Suitable buffers include, but are not limited to, organic acid salts such as salts of citric acid, ascorbic acid, gluconic acid, carbonic acid, tartaric acid, succinic acid, acetic acid, or phthalic acid; Tris, tromethamine hydrochloride, or phosphate buffers.

The formulation may also contain pharmaceutically acceptable carriers such as coloring agents, emulsifying agents, suspending agents, ethanol, EDTA or similar chelating agents, citrate buffer, water, and combinations thereof. Moreover, the compositions may include polymeric excipients/additives such as polyvinylpyrrolidones, ficolls, dextrates, polyethylene glycols, flavoring agents, anti-microbial agents, antioxidants, anti-static agents, steroids, and chelating agents.

The formulations of the present disclosure have good oral absorption properties. As used herein, the term “bioavailability” refers to the rate and amount of a substance that reaches the systemic circulation of a patient or subject following administration of the substance or form of the substance to the patient or subject. By definition, when a composition is administered intravenously, its bioavailability is 100 percent. However, when a composition is administered via other routes (such as orally), its bioavailability decreases (due to incomplete absorption and first-pass metabolism). More specifically, bioavailability is a measure of the ratio of the amount of substance “absorbed” from a test formulation to the amount “absorbed” after administration of a standard formulation. Frequently, the “standard formulation” used in assessing bioavailability is the aqueous solution of the substance, given intravenously.

Accordingly, bioavailability is one of the principal pharmacokinetic properties of substances and can be determined by evaluating, for example, the plasma or blood concentration-versus-time profile for a substance. Parameters useful in characterizing a plasma or blood concentration-versus-time curve include the area under the curve (AUC), the maximum concentration (Cₘₐₓ), and the time to maximum concentration (Tₘₐₓ). As used herein, the term “AUC” refers to the area under a curve representing the concentration of a compound or metabolite thereof in a biological fluid, e.g., plasma and blood, in a patient or subject as a function of time following administration of the compound to the patient or subject. The AUC may be determined by measuring the concentration of a compound or metabolite thereof in a biological fluid using methods such as liquid chromatography-tandem mass spectrometry (LC/MS/MS), at various time intervals, and calculating the area under the plasma concentration-versus-time curve. Suitable methods for calculating the AUC from a concentration-versus-time curve are well known in the art. Cₘₐₓ is the maximum concentration of a drug in the plasma or blood of a patient or subject following administration of a dose of the substance or form of the substance to the patient or subject. Tₘₐₓ is the time to the maximum concentration (Cₘₐₓ) of a substance in the plasma or blood of a patient or subject following administration of a dose of the substance or form of the substance to the patient or subject.

The amount of substance absorbed is taken as a measure of the ability of the formulation to deliver the substance to the sites of action. Obviously—depending on such factors as disintegration and dissolution properties of the dosage form, and the rate of biotransformation relative to the rate of absorption—dosage forms containing identical amounts of active substance may differ markedly in their abilities to make the substance available, and therefore, in their abilities to permit the substance to manifest its expected pharmacodynamic and therapeutic properties. The “amount absorbed” is conventionally measured by one of two criteria, either the area under the time-plasma concentration curve (AUC) or the total (cumulative) amount of the substance excreted in the urine following drug administration. A linear relationship exists between the AUC and dose when the fraction of the drug absorbed is independent of dose, and elimination rate (half-life) and volume of distribution are independent of dose and dosage form. However, when AUC is dependent on dose, as occurs when, for example, there is saturable absorption, significant metabolism, or poor solubility of the substance in the GI tract, a non-linear relationship exists between AUC and dose.

In order to assess the relative bioavailability of the formulations (and to correct for the slightly different doses administered with various forms due to the different molecular weights), the AUC plasma uptake values observed for the standard (here, creatine monohydrate) and each formulation are entered into the following equation to produce a ratio:

$$\frac{{AUC}_{SampleA} \times {Dose}_{B}}{{AUC}_{SampleB} \times {Dose}_{A}}$$

Based on this relationship, the relative bioavailability of any of the formulations to creatine monohydrate is about 1.3 or greater. In some embodiments, the relative bioavailability of any of the formulations to creatine monohydrate is about 1.5 or greater. In other embodiments, the relative bioavailability of any of the formulations to creatine monohydrate is about 1.6 or greater. In still other embodiments, the relative bioavailability of any of the formulations to creatine monohydrate is about 1.7 or greater.

In other words, the relative bioavailability of the formulations is at least about 30 percent greater than creatine monohydrate, about 40 percent greater than creatine monohydrate, or about 50 percent greater than creatine monohydrate. In one embodiment, the bioavailability of the formulations is at least about 50 percent greater than bioavailability of creatine monohydrate. In another embodiment, the bioavailability of the formulation is at least about 65 percent greater than bioavailability of creatine monohydrate. In yet another embodiment, the formulations have a bioavailability of at least about 70 percent greater relative to creatine monohydrate.

Based on this relationship, the relative bioavailability of any of the formulations to creatinine is about 2.0 or greater. In some embodiments, the relative bioavailability of any of the formulations to creatinine is about 2.2 or greater. In other embodiments, the relative bioavailability of any of the formulations to creatinine is about 2.4 or greater. In still other embodiments, the relative bioavailability of any of the formulations to creatinine is about 2.7 or greater.

The creatinine pronutrient and formulation may be produced in powder or crystal form. In one embodiment, the formulation is encapsulated or tableted for an oral dosage. For example, the formulation may be administered in the form of a pill, tablet, capsule, or gel capsule. In another embodiment, the formulation may be administered in a liquid form. For example, the formulation may be administered as an elixir.

Preparations of the formulations are preferably at least about 80 percent pure, preferably at least about 95 percent pure, more preferably at least about 97 percent pure, and even more preferably at least about 99 percent pure. The term “pure” as used herein refers to the lack of impurities or other constituents in the preparation.

In some embodiments, the formulation includes about 75 percent to about 90 percent creatinine pronutrient(s) and about 10 percent to about 25 percent creatinine. For example, the formulation may include about 75 percent to about 85 percent and about 15 percent to about 25 percent creatinine. In other embodiments, the formulation may include about 95 percent to about 100 percent creatinine pronutrient(s) and less than about 5 percent creatinine.

### Administration

In one embodiment, the formulation is administered to a patient. A patient may include, but is not limited to, a human, a canine, and an animal. Dosages range from use “as needed” to daily dosages of 1-2 doses taken 1-4 times daily. In this aspect, a formulation including an effective amount of the creatinine pronutrient may be administered once or twice daily. In another embodiment, the formulation containing an effective amount of the creatinine pronutrient may be administered multiple doses in a day, e.g., two to four doses. For example, in some embodiments, the formulation containing an effective amount of the creatinine pronutrient may be administered in three separate doses per day. In another embodiment, the formulation containing an effective amount of the creatinine pronutrient may be administered in four separate doses per day.

In this aspect, if the daily effective amount of creatinine pronutrient is from about 500 mg to about 1500 mg per 100 pounds of body weight, a patient or subject up to 250 pounds may receive one dose of the formulation containing about 1500 mg to about 3000 mg of the creatinine pronutrient daily or receive two separate doses of the formulation containing about 750 mg to about 1500 mg of the creatinine pronutrient. In another embodiment, if the daily effective amount of creatinine pronutrient is from about 500 to about 1500 mg per 100 pounds of body weight, a patient or subject up to 250 pounds may receive three separate doses of the formulation, each containing about 500 mg to about 1000 mg of creatinine pronutrient, per day. In yet another embodiment, if the daily effective amount of creatinine pronutrient is from about 500 to about 1500 mg per 100 pounds of body weight, a patient or subject up to 250 pounds may receive four doses of the formulation, each containing about 375 mg to 750 mg of creatinine pronutrient, per day.

The methods of administration of the composition described herein to the patient or subject may vary. However, in one embodiment, the formulation is administered to the patient or subject intravenously. In fact, as briefly discussed above, intravenous administration translates to 100 percent bioavailability and may be particularly suitable when the formulation is co-administered with other therapies and/or antivirals that are already administered intravenously. In this aspect, when administered intravenously, the creatinine pronutrient(s) may be present in an amount of up to about 10 mM. In some aspects, the creatinine pronutrient(s) is present in amount of about 0.1 mM to about 10 mM. In other aspects, the creatinine pronutrient is present in an amount of about 0.5 mM to about 8 mM. In one embodiment, the formulation is administered to a patient or subject with a need for adjusted blood creatinine levels intravenously for up to 5 days. In another embodiment, the formulation is administered to a patient intravenously for up to 10 days. In another embodiment, the formulation is administered to a patient intravenously for up to 15 days.

In another embodiment, the formulation is administered to the patient or subject orally. For example, the formulation may be encapsulated or tableted for a solid oral dosage form. In one embodiment, the formulation may be administered in the form of a pill, tablet, capsule, or gel capsule. In the alternative, the formulation may also be provided orally to the patient or subject in a liquid, gel, or powder form. For example, the formulation may be in the form of a powder suitable for mixing with water or other liquids. In this aspect, the formulation may be added into a beverage or may be provided as an ingredient premixed in a beverage. The formulation may also be administered as an elixir or as a solution formulation. In still another embodiment, the formulation may be administered in the form of a functional food, for example, a shake or bar.

In one embodiment, an effective oral dosage of the formulation ranges from about 400 mg to 2400 mg per day, or about 5 mg/kg to about 30 mg/kg. In another embodiment, an effective oral dosage ranges from about 400 mg to about 800 mg, or about 5 mg/kg to about 10 mg/kg. In yet another embodiment, an effective oral dosage ranges from about 400 mg to about 1200 mg, or about 5 mg/kg to about 15 mg/kg.

In some embodiments, the effective oral dosage of the formulation includes about 500 mg to about 2400 mg of the creatinine pronutrient. In other embodiments, the effective oral dosage includes about 750 mg to about 2000 mg of the creatinine pronutrient. In still other embodiments, the effective oral dosage includes about 1000 mg to about 1500 mg of the creatinine pronutrient. In yet other embodiments, the effective oral dosage of the formulation includes about 750 mg to about 1425 mg of the creatinine pronutrient and about 75 mg to about 600 mg of the second creatine derivative.

In still other embodiments, the formulation is administered to the patient or subject topically. Topical compositions including the formulations of the present disclosure may be in any form suitable for application to a body surface including, for example, ointment, cream, gel, lotion and paste forms, which may be formulated as an occlusive or semi-occlusive composition to provide enhanced hydration. Ointments are semi-solid preparations normally having a petrolatum (soft paraffin) or other petroleum derivative base, which is classified as either an oleaginous, emulsifiable, emulsion or water-soluble base. Creams are viscous liquids or semisolid emulsions, which may be oil-in-water or water-in-oil emulsions. Gels are semi-solid suspension systems that comprise an organic macromolecule distributed substantially uniformly throughout a liquid carrier medium, which is normally aqueous, but may also contain an alcohol and, optionally, an oil. Lotions are usually liquid or semi-liquid preparations in which solid particles are present in a water or alcohol base. Pastes are semi-solid carrier vehicles in which an active ingredient is suspended in a suitable base material, such as petrolatum, hydrophilic petrolatum or the like, which form a fatty paste. A paste may also be prepared from a single-phase aqueous gel of the type described above, using carboxymethyl cellulose or the like as a base material.

In this aspect, the one or more creatinine pronutrient(s) may be present in an amount of at least 0.5 percent and preferably from about 3 percent to about 99.5 percent, such percentages being based on the total weight of the composition. In some aspects, the one or more creatinine pronutrients is included in a topical formulation in an amount of about 5 percent to about 90 percent. In other aspects, the one or more creatinine pronutrients is included in a topical formulation in an amount of about 10 percent to about 95 percent. In still other aspects, the one or more creatinine pronutrients is included in a topical formulation in an amount of at least about 50 percent with the remainder a creatine or creatine derivative such as a creatine salt.

When used in an aqueous, topical form, the creatinine pronutrient(s) may be present in an amount of at least about 10 mM. In some aspects, the creatinine pronutrient(s) may be present in an amount of at least about 50 mM. In other aspects, the creatinine pronutrient(s) may be present in an amount of about 100 mM to about 2M. Anhydrous forms of the formulations of the present disclosure may include the creatinine pronutrient(s) in an amount of at least about 2 percent, at least about 10 percent, at least about 20 percent, or at least about 30 percent based on the total weight of the composition. In some aspects, anhydrous forms of the formulations of the present disclosure ma include the creatinine pronutrient(s) in an amount of about 10 percent to about 40 percent based on the total weight of the composition.

Administration of the formulation of the present disclosure has been found to increase acute blood levels of creatinine. In particular, administration of the formulation of the present disclosure results in the increase of acute blood levels of creatinine by at least about 10 percent compared to values prior to treatment. In particular, one embodiment contemplates an increase of acute blood levels of creatinine levels by about 10 percent to about 70 percent when patients receive a daily dose of at least about 5 mg/kg over a two-week period.

For example, the increase of intracellular creatinine levels may be about 10 percent or more at a creatinine pronutrient concentration level of about 20 μM or more. In another embodiment, the effects of creatinine pronutrient at a concentration of about 20 μM or more result in an increase of creatinine levels of about 15 percent or more. In yet another embodiment, the effects of a formulation with the creatinine pronutrient at a concentration of about 20 μM or more result in an increase of creatinine levels of about 20 percent or more. For example, a concentration of about 20 μM or more of creatinine pronutrient may result in an increase of creatinine of at least 25 percent or more, about 30 percent or more, about 35 percent or more, and about 40 percent or more. In this aspect, the increase in intracellular creatinine levels would extend to any type of cell including, but not limited to endothelial, epithelial, immune, neutron, and microglia cells.

When the concentration of the creatinine pronutrient is about 100 μM or more, the increase of intracellular creatinine about 1 to 4 hours after administration is about 30 percent or more as compared to creatinine levels in the blood prior to the administration of the formulation. In another embodiment, the effects of a formulation including the creatinine pronutrient at a concentration of about 100 μM or more result in an increase of intracellular creatinine about 1 to 4 hours after administration of about 35 percent or more. For example, a concentration of about 100 μM or more of creatinine pronutrient may result in an increase of intracellular creatinine about 1 to 4 hours after administration of at least 40 percent or more, about 45 percent or more, and about 50 percent or more.

Without being bound to any particular theory, since the creatinine pronutrient increases intracellular creatinine, there is also an increase in the intracellular concentration of creatinine metabolites (typically about 1 to 6 hours after administration). In this aspect, when the concentration of the creatinine pronutrient is about 100 μM or more, there is an increase in concentration of intracellular creatinine metabolites about 1 to 6 hours after administration of about 20 percent or more as compared to the concentration of intracellular creatinine metabolites prior to the administration of the formulation. In another embodiment, when the concentration of the creatinine pronutrient is about 100 μM or more, there is an increase in concentration of intracellular creatinine metabolites about 1 to 6 hours after administration of about 30 percent or more as compared to the concentration of intracellular creatinine metabolites prior to the administration of the formulation.

In some embodiments, the administration of the formulation in adults (i.e., 18 and older) of the present disclosure raises creatinine levels about 1 to 4 hours after administration in patients with below-normal creatinine levels to between about 0.5 and 1.4 mg/dL. More specifically, normal creatinine levels, i.e., about 0.5 to 1.1 mg/dL in healthy adult females and 0.6 to 1.35 mg/dL in healthy adult males, may be achieved about 1 to 4 hours after administration of the formulation of the present disclosure in populations that typically have below-normal creatinine levels such as the elderly, pregnant women, those suffering from muscle-wasting diseases, malnutrition, and chronic illness (such as liver disease), vegetarians, and the like. Without being bound by any particular theory, the formulations of the present disclosure allow persons with creatinine levels that are considerably lower than the average value for that person's age and gender to achieve normal creatinine levels. This is a surprising and unexpected result. Indeed, as accepted by the medical community, such populations have decreased muscle mass, which leads to less creatinine produced in the muscles and less creatinine present in the blood. However, even when the body is incapable of producing sufficient creatinine due to age, loss of muscle mass, or diet restrictions, administration of the formulation of the present disclosure increases the creatinine levels in the blood and allow the body to achieve normal creatinine levels.

The creatinine clearance, reported as milliliters of creatinine per minute per body surface area (mL/min/BSA), may also be used to evaluate creatinine levels in the body. Without being bound by any particular theory, since creatinine usually enters and is filtered from the bloodstream at a generally constant rate, the amount of creatinine in measured in the blood and urine should be relatively stable for a person with normal creatinine levels. In particular, creatinine is transported through the bloodstream to the kidneys and the kidneys filter out most of the creatinine and dispose of it in urine (provided that the kidneys have normal excretory function). As such, measuring urine creatinine levels may demonstrate the increased production of creatinine in the body after administration of the formulations of the present disclosure. In this regard, administration of the formulations of the present disclosure may facilitate/stabilize creatinine clearance levels in male patients of any age to about 70 to about 170 mL/min/BSA. While the creatinine clearance levels in female patients typically vary more by age than in male patients, administration of the formulations of the present disclosure may facilitate/stabilize creatinine clearance levels of about 75 to about 165 mL/min/BSA for 18 to 29 year old females, about 70 to about 160 mL/min/BSA for 30 to 39 year old females, about 65 to about 146 mL/min/BSA for 40 to 49 year old females, about 60 to about 140 mL/min/BSA for 50 to 59 year old females, and about 50 to about 135 mL/min/BSA for 60 to 72 year old females.

Although the benefits of administration of the formulations as described herein largely focus on treating a patient with below-normal creatinine levels, the present invention contemplates the use of the formulations of the present disclosure to maintain normal creatinine levels for patients that may be suffering from a short-term decrease in creatinine levels. For example, the formulations of the present disclosure may be useful to maintain healthy/normal creatinine levels in a person that is fasting or dieting. In addition, since the creatinine pronutrients have been demonstrated to prevent and/or treat inflammation and/or boosting antiviral immune activity, the formulations of the present disclosure may provide the dual/triple benefit of increasing blood creatinine levels, preventing/treating inflammation, and increasing immune activity.

Furthermore, the creatinine pronutrients of the present disclosure may be useful to reduce loss of integrity in the blood-brain barrier, which may be particularly useful when a subject is undergoing stress, cancer immunotherapy, or other conditions, disease states, or other therapy. For example, the creatinine pronutrients and formulations including the creatinine pronutrients of the present disclosure may be useful to inhibit or reduce the increases in the permeability coefficient. In some aspects, administration of a formulation including at least one creatinine pronutrient of the present disclosure may reduce the permeability coefficient after exposure to or triggering of low concentration (about 10 nM to about 1 μM) inflammatory stimuli by about 30 percent to about 70 percent (as compared to no treatment with a formulation of the present disclosure). In other aspects, administration of a formulation including at least one creatinine pronutrient of the present disclosure may reduce the permeability coefficient after exposure to or triggering of low concentration (about 10 nM to about 1 μM) inflammatory stimuli by about 40 percent to about 60 percent (as compared to no treatment with a formulation of the present disclosure). In still other aspects, administration of a formulation including at least one creatinine pronutrient of the present disclosure may reduce the permeability coefficient after exposure to or triggering of high concentration (about 10 μM or more) inflammatory stimuli by about 30 percent to about 70 percent (as compared to no treatment with a formulation of the present disclosure). In yet other aspects, administration of a formulation including at least one creatinine pronutrient of the present disclosure may reduce the permeability coefficient after exposure to or triggering of high concentration (about 10 μM or more) inflammatory stimuli by about 40 percent to about 60 percent (as compared to no treatment with a formulation of the present disclosure).

Without being bound to any particular theory, such increases in the permeability coefficient (and loss of blood-brain barrier integrity) are likely due to inflammatory stimuli such as Bradykinin. Accordingly, without being bound by any particular theory, the creatinine pronutrients and formulations including such creatinine pronutrients of the present disclosure may also be useful in such subjects by reducing or inhibiting activation or signaling of the BK receptors. As such, the optimized creatinine levels achieved through the administration of the formulations of the present disclosure may also reduce or inhibit Bradykinin receptor activation and/or signaling, reduce or prevent an increase in calcium mobilization in the cell, and/or reduce or prevent generation of nitro oxide, which, in turn, reduces or prevents increases in permeability.

In addition, the formulations of the present disclosure may also reduce or inhibit generation of prostaglandins (PGE 2 and PGF) when exposed to inflammatory stimuli such as Bradykinin (which usually can be observed in vitro within about 15 minutes and reaching maximum release within several hours). For example, administration of a formulation including at least one creatinine pronutrient of the present disclosure may reduce or inhibit PGE release better than known BK receptor 2 antagonist HOE. In some aspects, administration of a formulation including at least one creatinine pronutrient of the preset disclosure may reduce or inhibit PGE release (pg/mg protein) 6 hours after exposure to or triggering of inflammatory stimuli (such as Bradykinin) by about 30 percent to about 70 percent (as compared to no treatment with a formulation of the present disclosure). In other aspects, administration of a formulation including at least one creatinine pronutrient of the present disclosure may reduce or inhibit PGE release (pg/mg protein) 6 hours after exposure to or triggering of inflammatory stimuli (such as Bradykinin) by about 40 percent to about 60 percent (as compared to no treatment with a formulation of the present disclosure). In still other aspects, administration of a formulation including at least one creatinine pronutrient of the present disclosure may reduce or inhibit PGE release (pg/mg protein) 6 hours after exposure to or triggering of inflammatory stimuli (such as Bradykinin) by about 45 percent to about 55 percent (as compared to no treatment with a formulation of the present disclosure).

In yet other aspects, administration of a formulation including at least one creatinine pronutrient of the present disclosure may reduce or inhibit PGE release (pg/mg protein) 24 hours after exposure to or triggering of inflammatory stimuli (such as Bradykinin) by about 30 percent to about 70 percent (as compared to no treatment with a formulation of the present disclosure). In other aspects, administration of a formulation including at least one creatinine pronutrient of the present disclosure may reduce or inhibit PGE release (pg/mg protein) 24 hours after exposure to or triggering of inflammatory stimuli (such as Bradykinin) by about 40 percent to about 60 percent (as compared to no treatment with a formulation of the present disclosure). In still other aspects, administration of a formulation including at least one creatinine pronutrient of the present disclosure may reduce or inhibit PGE release (pg/mg protein) 24 hours after exposure to or triggering of inflammatory stimuli (such as Bradykinin) by about 45 percent to about 55 percent (as compared to no treatment with a formulation of the present disclosure).

### EXAMPLES

The following non-limiting examples are merely illustrative of the preferred embodiments of the present invention, and are not to be construed as limiting the invention, the scope of which is defined by the appended claims.

### Example 1: Formulations

Several example formulations made in accordance with the present disclosure are provided below:

TABLE 1
Example Formulations

| | Dose (mg per capsule or scoop) | | | | | | | | |
|---|---|---|---|---|---|---|---|---|---|
| Ingredient | Ex A | Ex B | Ex C | Ex D | Ex E | Ex F | Ex G | Ex H | Ex I |
| Creatinine | 750 mg | — | 750 mg | 1000 mg | 500 mg | 1000 | 750 mg | — | 500 mg |
| Pronutrient 1 | | | | | | | | | |
| Creatinine | — | 750 mg | 750 mg | 500 mg | 1000 mg | 1000 mg | — | 750 mg | 500 mg |
| Pronutrient 2 | | | | | | | | | |
| Creatine | — | — | — | — | — | — | 750 mg | 750 mg | 500 mg |
| additive | | | | | | | | | |

The formulation may include one or more creatinine pronutrient disclosed herein or one or more creatinine pronutrient and another form of creatine. For example, the creatinine pronutrient 1 and/or creatinine pronutrient 2 in Table 1 above may be ethyl (α-guanido-methyl) ethanoate, creatine ethyl ester hydrochloride (CEE), creatine isopentyl ester, creatine tert-butyl ester (CtB), creatine cyclohexyl ester, creatine glycinamide ethyl ester, or salts, derivatives, or combinations thereof. The creatine additive may be creatine hydrochloride, creatine glycerol laurate ester, creatine propylene glycol laurate ester, creatine amide tert butyl ester, dicreatine glycerol ester, tricreatine glycerol ester, betaine cyclopentyl ester, creatine mesylate, creatine citrate, creatine pyruvate, and the like.

The formulations may be in capsule or powder form. For instance, Examples A and B may be administered once a day with a serving size of 2 capsules (750 mg per capsule). Examples A and B may also be formulated in powder form and be administered twice a day (750 mg per scoop). Examples C-I may be administered once a day by mixing one or two scoops with 8-12 ounces of water or other liquid suitable for ingestion. Examples C-I may also be formulated in capsule and taken once a day.

### Example 2: Solubility Determination

The saturated solubility of creatinine pronutrients of Example 1 in deionized water was determined by adding increasing amounts to 5 ml of solvent in screw-capped glass bottles placed in a shaking water bath at 25° C. After 1.5 hour, the saturated solutions were vortexed and 2-ml aliquots removed and centrifuged in microcentrifuge tubes at 11,000 rpm for 5 min. Concentrations were analyzed by HPLC by diluting 500 μL of supernatant with mobile phase (500 μL). The mean±standard deviation of the saturation solubility for each antiviral therapeutic compound is calculated from the corresponding standard curves.

As shown in Table 2 below, the solubility of ethyl (α-guanido-methyl) ethanoate, creatine ethyl ester hydrochloride, and creatine isopentyl ester salt are significantly higher than creatine monohydrate. In the embodiment used to generate the data in Table 2, the salt (A) that is combined with creatine cyclohexyl ester is tosylate, which is not a particularly strong counter ion. As such, it is believed that the use of HCl or Br salts in place of the tosylate may result in higher solubility.

TABLE 2
Aqueous Solubility Assessments

| | | Ethyl (α- | | Creatine | Creatine | Creatine | Creatine |
|---|---|---|---|---|---|---|---|
| | Control | guanido- | Creatine | Isopentyl | Glycinamide | Cyclohexyl | Tert- |
| | (Creatine | methyl) | Ethyl | Ester | Ethyl Ester | Ester | Butyl |
| | Monohydrate) | ethonoate | Ester | HCl | HCl | Tosylate | Ester HCl |
| Solubility | 21.0 ± 1.9 | 178 ± 7.2 | 205 | >600 | >600 | 15-20 | 389 +/− 11 |
| (mg/ml) | | | | | | | |
| Ratio | 1.0 | >8 | >9 | >29 | >29 | <1.0 | ≥18 |
| (relative to | | | | | | | |
| CM) | | | | | | | |

### Example 3: Creatinine Uptake Following Exposure to Creatinine Pronutrients

In an effort to determine the impact/effectiveness of a formulation made in accordance with the present disclosure on increasing creatinine delivery, different cell types were exposed to several of the formulations in Example 1. More specifically, cellular uptake studies were performed for formulations including CEE and CtB from Example 1.

The cell lines used in this example were chosen to provide a broad range of cells (including kidney tubule epithelial cells that represent cells with potentially high transport, endothelial cells that represent the biological interface between blood and extracellular tissue, macrophage cells representing immune cells that would be present in the blood, myocardial cells representing muscle cells that would produce creatinine through normal cardiac activity):
human astrocytes (HA)
mouse macrophage cell line (ANA-1)
human cardiomyocyte cell line (HL-1)
human colon epithelial (Caco-2)
Canine kidney epithelial (MDR)
human brain endothelial cells (HCMEC/d3)
   For instance, based on earlier studies by Ku and Passow (1980), which found a linear uptake of creatinine in red blood cells, it is believed that passive diffusion processes drive creatinine uptake (as compared to creatine, which has a saturable and transport-dependent uptake). In other words, it remains unknown where there is a transporter involved in the accumulation of creatinine. Without being bound by any particular theory, if a transporter is involved for accumulation of creatinine, it may be the organic cation transporter 2 (OCT2) found in kidney tubule epithelial cells.

### Study Design and Results

The various cell lines discussed above were first exposed to 1 mM of creatinine (CRN), CEE, and CtB at 37° C. for a 3-hour incubation period at a pH 7.4. After incubation, the assay buffer was collected. The cells were then washed rapidly three times with ice-cold phosphate-buffered saline solution. After washing, cells were lysed with a lysis buffer and samples of the cell lysate were used to determine intracellular creatinine levels in each cell type (separate cell lysate samples were used to determine protein content of cell lysates).

More specifically, the creatinine content in cell lysates was determined using Quantichrom™ Creatinine Assay Kit (DICT 500) from Bioassay Systems (Hayward, CA). The assay quantitatively measures creatinine without any pretreatment through an optimized Jaffe method. The Jaffe method uses picrate that forms a red colored complex with creatinine. The intensity of the red, measured at 510 nm, is directly proportional to the concentration of creatinine in the sample.

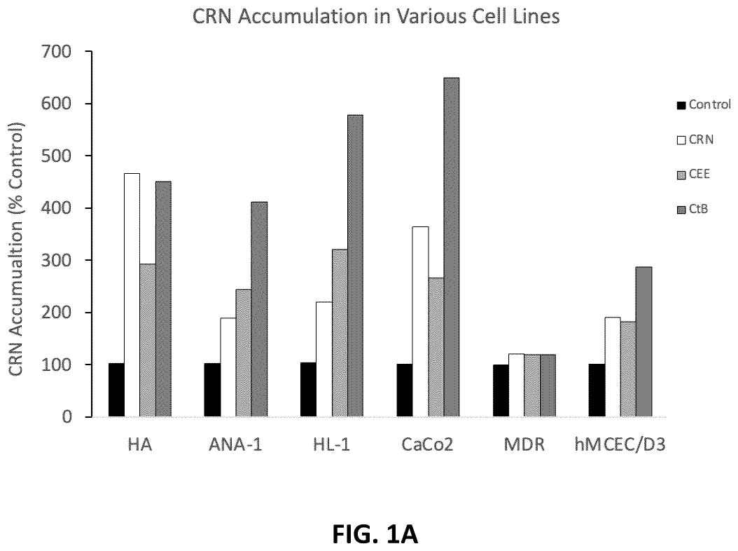

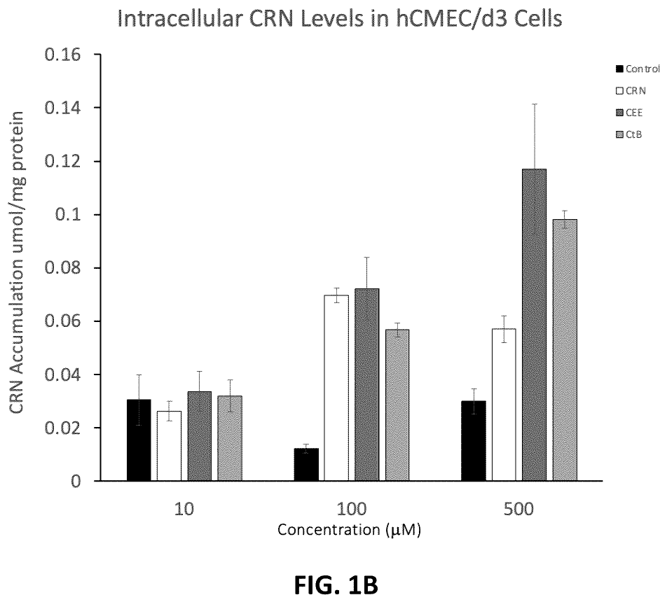

Creatinine accumulation in the various cell lines is shown in FIG. 1A. Values indicate increases in creatinine accumulation in cells compared to control groups receiving only assay buffer and represent the mean of 3 cell monolayers per treatment group. In all cell lines and creatinine pronutrients studied, the creatinine pronutrients increased intracellular levels of creatinine. In the case of at least ANA-1 and HL-1, the creatinine accumulation after a 3-hour period was greater with 1 mM of the creatinine pronutrient than 1 mM creatinine. In the case of Caco-2 and HCMEC/d3, creatinine accumulation after a 3-hour period of exposure to 1 mM of CtB was greater than the creatinine accumulation after a 3-hour period of exposure to 1 mM creatinine. In fact, in Caco-2, the creatinine accumulation after a 3-hour period of exposure to 1 mM CtB was almost double that of the creatinine accumulation after a 3-hour period of exposure to 1 mM creatinine.

In an effort to determine whether there is a saturable transport dependent uptake mechanism involved, HCMEC/d3 cells were exposed to the following range of concentrations for these formulations for a 3-hour incubation period at 37° C. for a 3-hour incubation period at a pH 7.4:
10 μM
100 μM
500 μM
   The impact of the different formulations and concentrations on intracellular creatinine levels in HCMEC/d3 were examined by comparing maximal increases in total intracellular creatinine (mmol/mg protein) in treated cells as compared to cells receiving assay buffer alone (control). As shown in FIG. 1B, at higher concentrations (500 μM), the creatinine pronutrients can increase intracellular levels of creatinine better than with creatinine exposure.

### Example 4: Plasma Creatinine Levels Following Creatinine Pronutrient Dosing

A rat PK study was performed to determine resulting plasma creatinine levels following creatinine pronutrient dosing. In this study, a 10 mg/kg oral (low) dose, a 70 mg/kg oral (high) dose, and 10 mg/kg IV dose of ¹³C-labeled creatine monohydrate and creatinine pronutrient were administered. Levels of creatine, creatinine pronutrient, and creatinine in the plasma were measured over time.

### Study Design

The study used twenty-four (24) sprague-dawley male rats separated into 6 groups as shown in Table 3 below. In this study, the specific creatinine pronutrient used was creatine ethyl ester hydrochloride.

TABLE 3
Rat pK Study Groups
Measurement of Creatine, Creatinine Pronutrient, and Creatinine Levels

| Dose | Creatine Monohydrate | Creatinine Pronutrient |
|---|---|---|
| 10 mg/kg (oral) | [symbol] | [symbol] |
| 70 mg/kg (oral) | [symbol] | [symbol] |
| 10 mg/kg (IV) | [symbol] | [symbol] |

Blood samples were collected over a 4-hour period via femoral artery catheter. Muscle and brain tissue were collected at the end of the study. Liquid chromatography-mass spectrometry (LCMS) was used for analysis of samples. The area under the plasma concentration curve following oral dosing was compared to that obtained following IV administration.

### Results

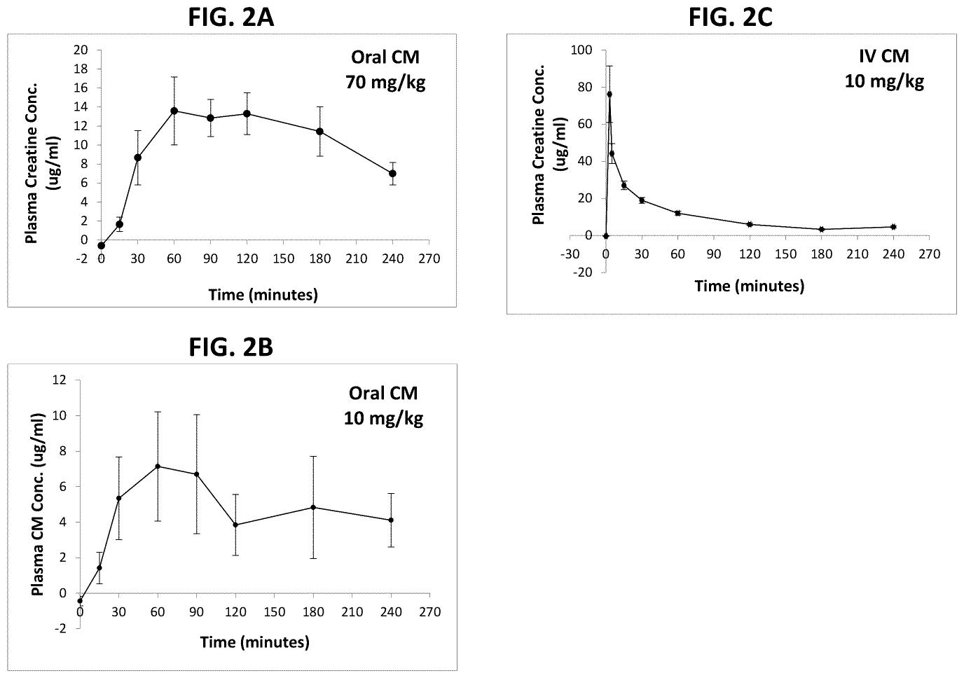

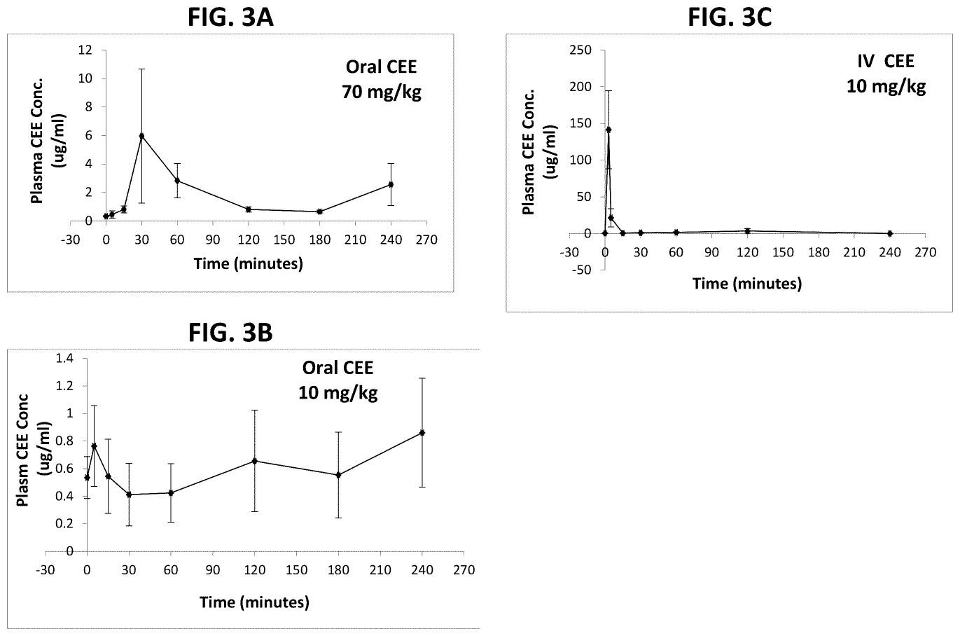

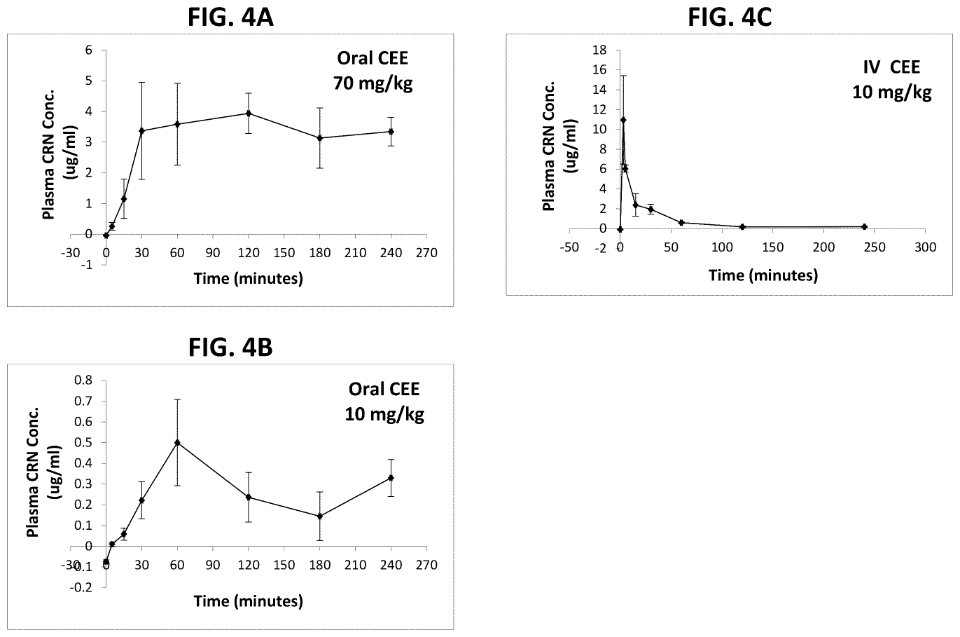

Following the administration of the creatinine pronutrient, no detectable amount of creatine was measured in the plasma samples. Small but measurable amounts of creatinine pronutrient were present. Substantial levels of creatinine were present in the plasma samples (especially at the high dose). More specifically, FIGS. 2A-2C shows creatine levels after dosing with creatine monohydrate, FIGS. 3A-3C show creatinine pronutrient levels after dosing with the creatinine pronutrient, and FIGS. 4A-4C show creatinine levels after dosing with the creatinine pronutrient.

### Example 5: Creatinine Pronutrient Bioavailability

Bioavailability of creatinine and the creatinine pronutrient is shown in Table 4 below.

TABLE 4
Bioavailability of Creatinine and Creatinine Pronutrient

| | | | | | | Relative |
|---|---|---|---|---|---|---|
| | Dose | Cₘᵢₙ | Cₘₐₓ | Tₘₐₓ | | Bio- |
| | Administered | (μg/ml) | (μg/ml) | (min) | AUC₍₀₋₁₂₀ₘ₎ | availability |
| Creatinine | 3 grams | 11.3 | 15.3 | 60 | 276 | 1.0 |
| Creatinine | 1.5 grams | 0 | 10 | 120 | 372 | 2.70 |
| Pronutrient | 0.18 gram | 0 | 0.5 | 60 | 35.5 | 2.14 |
| Formulation | | | | | | |

The creatinine data in Table 4 was generated from the data provided in Ostojic et al., Food Sci Nutr. 2019. In this 2019 study, human subjects were administered one of three dietary formulations: 1) creatine nitrate (3 grams); 2) creatine nitrate+creatinine (3 grams each); and 3) creatine monohydrate (3 grams) and single oral dose pharmacokinetics measured both creatine and creatinine plasma levels. Using the second formulation, i.e., the creatine nitrate and creatinine (3 grams each), the area under the plasma concentration curve was computed from 0-120 minutes (AUC₀₋₁₂₀) along with the minimum and maximum creatinine concentrations (Cₘᵢₙ, Cₘₐₓ) observed and the time point at which Cₘₐₓ was observed (Tₘₐₓ).

The creatinine pronutrient formulation data in Table 4 was generated from the creatinine data gathered in Example 4 using allometric dosing to determine the human dose equivalent for the labeled creatinine pronutrient (for a 70 kg human).

In order to assess the relative bioavailability of the formulations (and to correct for the slightly different doses administered with various forms due to the different molecular weights), the AUC plasma uptake values observed for creatinine and the creatinine pronutrient formulation were entered into the following equation to produce a ratio:

$$\frac{{AUC}_{SampleA} \times {Dose}_{B}}{{AUC}_{SampleB} \times {Dose}_{A}}$$

Using the equation above, the relative bioavailabilities for both the high and low doses of the creatinine pronutrient formulation was compared to creatinine. As shown in Table 4, the creatinine pronutrient formulations deliver twice as much creatinine to the plasma as compared to creatinine.

### Example 6: Permeability Studies

In an effort to determine the impact/effectiveness of creatinine pronutrient formulations made in accordance with the present disclosure, studies examined bradykinin-induced effects on endothelial permeability using a microfluidic blood-brain barrier (BBB) culture model consisting of hCMEC/d3 (human brain endothelial cells) grown on Mimeta microfluidic plates. More specifically, permeability studies were performed using the microfluidic BBB culture model under control conditions and following exposure to a low dose (10 nM) or high dose (10 μM) of bradykinin in either the presence or absence of creatine ethyl ester hydrochloride (CEE) as the creatinine pronutrient. The addition of CEE to the microfluidic BBB culture model resulted in reduced permeability responses to bradykinin.

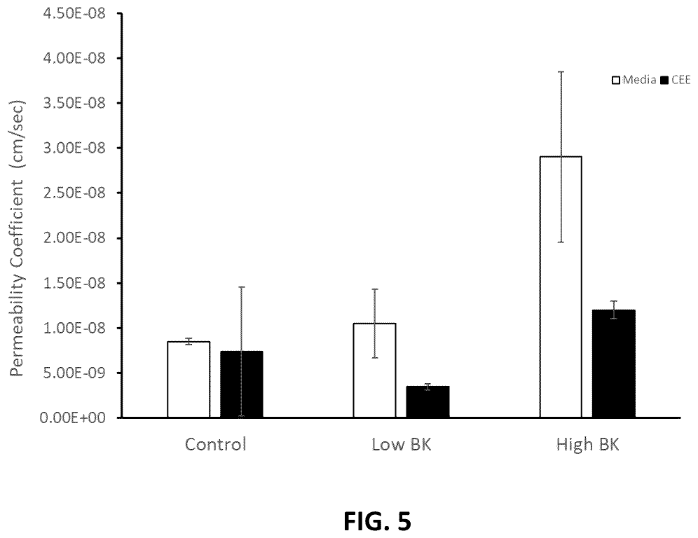

hCMEC/d3 cells in Mimeta microfluidic plate were pretreated for 1 hour with media or media+1 mM CEE. Thereafter, the cells were treated with a low concentration (10 nM) or high concentration (10 μM) of bradykinin. The permeability of a fluorescent dye (IRdye 800) was assessed after 30 minutes. As shown in FIG. 5, treatment with 10 nM bradykinin resulted in a slight increase in the permeability of IRdye 800 compared to control. Treatment with 10 μM bradykinin caused an even greater increase in permeability with an approximately 2-fold increase over control group. The increased leakage of fluorescent dye across the microfluidic BBB culture model following bradykinin exposure is consistent with the known effects of this proinflammatory agent. However, in the presence of the creatinine pronutrient, the permeability enhancing effects of bradykinin are significantly reduced. When exposed to low concentration bradykinin (10 nM), the permeability coefficient dropped from 1.05×10⁻⁸ cm/s to 3.43×10⁻⁹ cm/s following pretreatment of the cells with 1 mM CEE for 1-hour. Likewise, when exposed to high concentration Bradykinin (10 μM), the permeability coefficient drops from 2.90×10⁻⁸ cm/s to 1.42×10⁻⁸ cm/s if the cells are pretreated with 1 mM CEE for 1-hour.

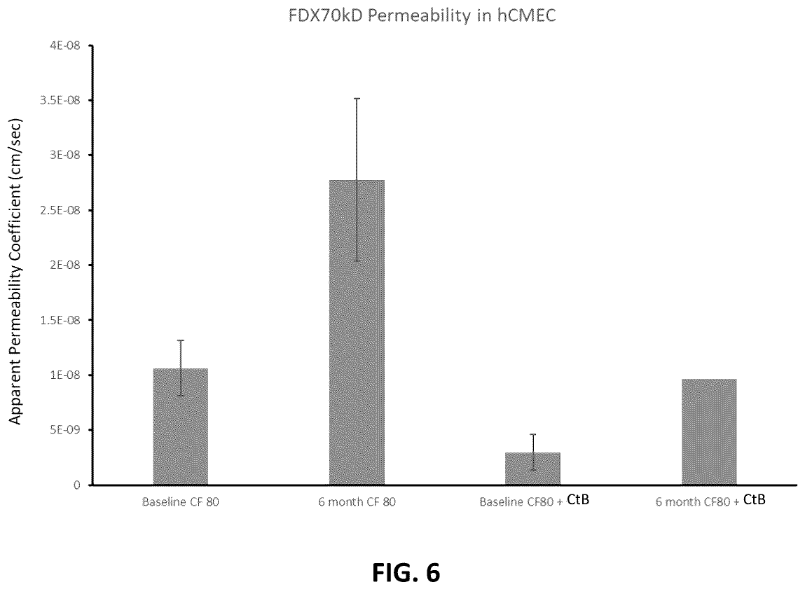

As increases in permeability in the microfluidic BBB model are representative of inflammatory effects on the brain endothelial cells, the model was used to identify pro-inflammatory responses to plasma samples taken from cancer patients undergoing treatment with doxorubicin. As shown in FIG. 6, 24-hour treatment of hCMEC/d3 microfluidic culture model with cancer patient plasma samples increases the permeability coefficient for both the large molecular weight fluorescent marker (Fluorescein labeled dextran; 70 kD) and the small molecular weight fluorescent marker (IRdye 800; 800 D). In some patients, significant increases in permeability were observed after 4 and 6-months of chemotherapy compared to baseline plasma samples-indicative of proinflammatory conditions produced by the chemotherapeutics. However, the permeability increases produced by the plasma samples from cancer patients were attenuated by the addition 1 mM creatinine pronutrient (creatine tert-butyl ester hydrochloride (CtB). Without any creatinine pronutrient, the permeability coefficient in breast cancer patient CF80 increased from 1.06×10⁻⁸ cm/s at baseline to 2.78×10⁻⁸ cm/s at 6 months. With CtB, the permeability coefficient in breast cancer patient CF80 was not only lower for the baseline plasma samples (2.97×10⁻⁹ cm/s) as compared to baseline in the media only samples, the permeability for the 6 month patient plasma samples was 9.63×10⁻⁹ cm/s in the CtB treatment group, which was significantly lower than the permeability coefficient at 6 months without CtB.

### Example 7: Prostaglandin Release Studies

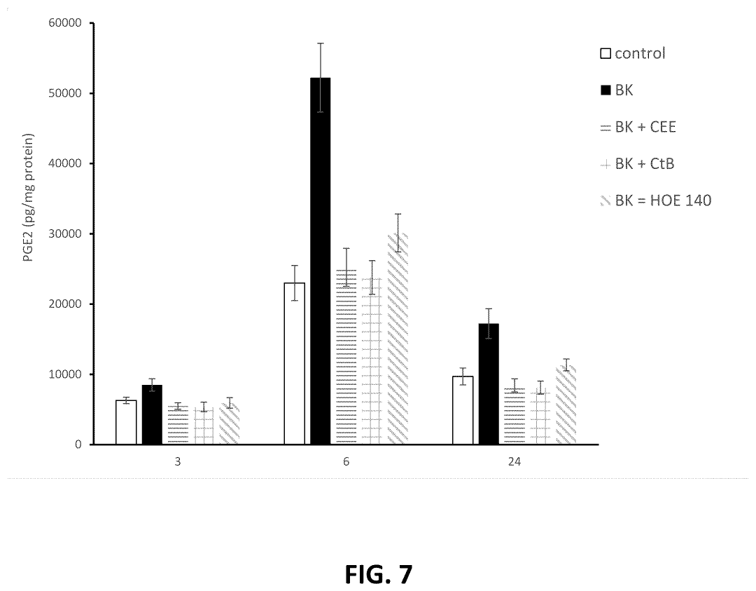

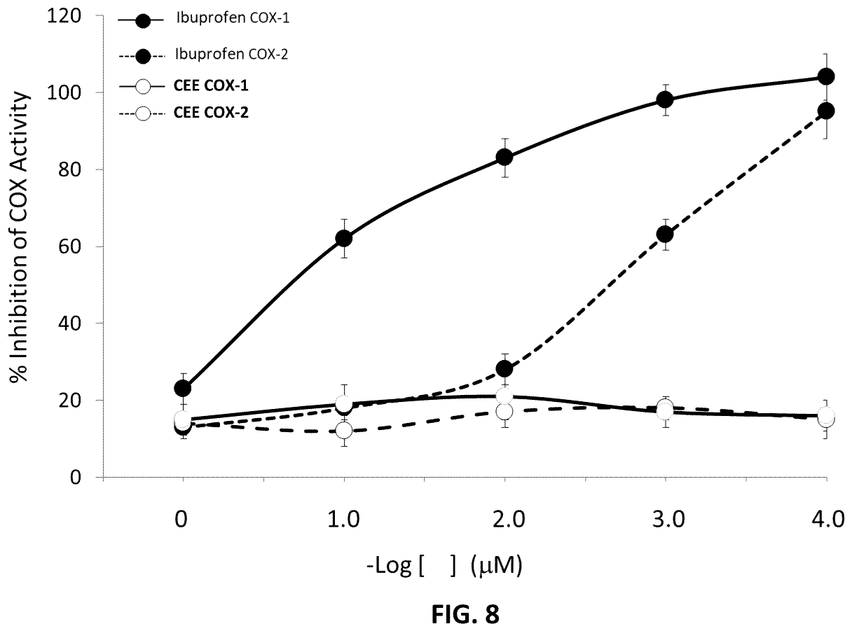

In an effort to determine the impact/effectiveness of creatinine pronutrient formulations made in accordance with the present disclosure on decreasing prostaglandin release over a 24-hour period due to bradykinin exposure, hBMEC/d3 cells were pre-treated with a creatinine pronutrient of the present disclosure and then exposed to 10 μM bradykinin. More specifically, hBMEC/d3 cells were grown on T25 flasks to confluency. Media was removed and exchanged with endothelial cell media containing 0.1% BSA and (1) 1.0 mM CEE, (2) 1.0 mM creatine t-butyl ester hydrochloride (CtB), or (3) 10 μM HOE 140 (a known BR2 receptor antagonist). After a 1-hour incubation, bradykinin (10 μM) was added and samples of media removed and assayed for prostaglandin E2 (PGE2) at 3, 6 and 24 hours. The amount of PGE 2 secreted into the media by the cells was determined using a PGE2 ELISA and normalized to cell lysate protein content. As shown in FIG. 7, the creatinine pronutrients were as effective at inhibiting prostaglandin release as the known BR2 receptor antagonist HOE 140 at 6 and 24 hours.

The effects of the creatinine pronutrient on PGE2 release was not due to the inhibition of prostaglandin synthesis through cyclooxygenase (COX) as production of prostaglandins by either COX1 or COX2 was unaffected by CEE. More specifically, as shown in FIGS. 7 and 8, while CEE prevents PGE release in response to the inflammatory stimuli, bradykinin, it does not do so in the same manner as an NSAID/a known COX inhibitor.

### Example 8: Inflammatory Stimuli Skin Prick Test

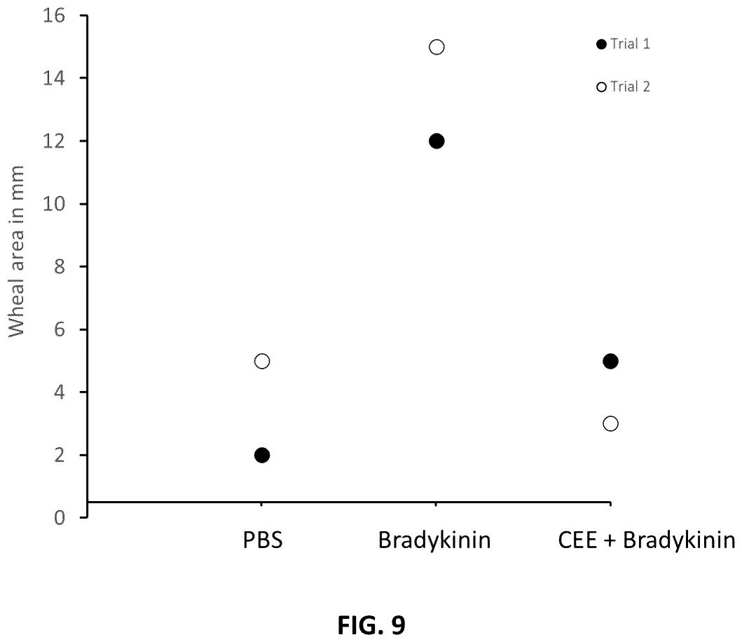

In order to determine whether topical administration of a creatinine pronutrient of the present disclosure was effective, a bradykinin skin test was conducted to measure wheal diameter with and without pretreatment with CEE. As shown in FIG. 9, a topical formulation including CEE reduced wheal formation (when exposed to bradykinin). In particular, different areas of the patient's right forearm was treated as follows: area 1—0.1 ml treatment solution consisting of phosphate buffered saline (PBS) alone; area 2—0.1 ml treatment solution consisting of 20 nM bradykinin; area 3-CEE topical ointment pretreatment, followed by 0.1 ml treatment solution consisting of 20 nM bradykinin. More specifically, area 3 was swabbed with the topical CEE ointment 15 minutes prior to exposure to treatment solution. The skin was then cleansed with ethanol solution and allowed to dry. The various treatment solutions were applied to the skin and the skin was then pricked through the solutions using a lancet and the area of wheal was measured at 20-30 minutes. As demonstrated in FIG. 9, topical CEE ointment pretreatment resulted in a decrease in wheal area produced by bradykinin to a size that was comparable to that observed with PBS alone.

Although the present invention has been described with reference to increasing intracellular and blood creatinine levels, it will be understood to those skilled in the art that the invention is capable of a variety of alternative embodiments within the spirit of the appended claims. For example, the formulations and method of the present disclosure are also contemplated for use to dampen the innate immune response, suppress bacterial replication, and/or attenuate the effects of pain and inflammation through anti-inflammatory immunomodulatory response.

What is claimed is:

1. A method for increasing extracellular creatinine levels in an animal, the method comprising:
providing a formulation, the formulation comprising a therapeutically effective dose of at least one creatinine pronutrient; and
administering a therapeutically effective dose of the formulation to the animal.

2. The method of claim 1, wherein the creatinine pronutrient comprises ethyl (α-guanido-methyl) ethanoate, creatine alkyl ester, creatine amide alkyl ester, creatine cyclohexyl ester, or salts, or derivatives or combinations thereof.

3. The method of claim 1, wherein the creatinine pronutrient comprises ethyl (α-guanido-methyl) ethanoate, creatine ethyl ester hydrochloride, creatine isopentyl ester hydrochloride, or derivatives or combinations thereof.

4. The method of claim 1, wherein the creatinine pronutrient comprises creatine isopentyl ester hydrochloride, or derivatives or combinations thereof.

5. The method of claim 1, wherein the formulation comprises about 30 to about 100 weight percent of the creatinine pronutrient.

6. The method of claim 1, wherein the creatinine pronutrient is present in the formulation at a concentration of about 0.1 mM to about 10 mM.

7. A method for increasing intracellular creatinine levels in an animal, the method comprising:
providing a formulation, the formulation comprising at least one creatinine pronutrient; and
administering a therapeutically effective dose of the formulation to the animal.

8. The method of claim 7, wherein the creatinine pronutrient comprises creatine tert-butyl ester hydrochloride or derivatives thereof.

9. The method of claim 7, wherein the formulation comprises about 30 to about 100 weight percent of the creatinine pronutrient.

10. The method of claim 7, wherein the creatinine pronutrient is present in the formulation at a concentration of about 0.1 mM to about 10 mM.

11. An oral supplement comprising a formulation comprising a creatinine pronutrient suitable for increasing creatinine levels in an animal, wherein the creatinine pronutrient comprises creatine alkyl ester, creatine amide alkyl ester, creatine cyclohexyl ester and salts, derivatives, or combinations thereof, and wherein the creatinine pronutrient is present in the formulation at a concentration of about 0.1 mM to about 10 mM.

12. The oral supplement of claim 11, wherein the formulation further comprises a creatine derivative.

13. The oral supplement of claim 11, wherein the creatinine pronutrient has a bioavailability that is at least 2 times the bioavailability of creatinine.

14. The oral supplement of claim 11, wherein the creatinine pronutrient comprises ethyl (α-guanido-methyl) ethanoate, creatine ethyl ester hydrochloride, creatine isopentyl ester hydrochloride, creatine tert-butyl ester hydrochloride, or derivatives or combinations thereof.

15. The oral supplement of claim 14, wherein the creatinine pronutrient comprises creatine isopentyl ester hydrochloride or derivatives thereof.

16. The oral supplement of claim 14, wherein the creatinine pronutrient comprises creatine tert-butyl ester hydrochloride or derivatives thereof.

17. A method for reducing permeability increases across the blood-brain barrier in an animal due to bradykinin release, the method comprising:
providing a formulation, the formulation comprising at least one creatinine pronutrient at a concentration of about 0.1 mM to about 10 mM; and
administering a therapeutically effective dose of the formulation to the animal.

18. The method of claim 17, wherein the creatinine pronutrient comprises ethyl (α-guanido-methyl) ethanoate, creatine ethyl ester hydrochloride, creatine isopentyl ester hydrochloride, creatine tert-butyl ester hydrochloride, or derivatives or combinations thereof.

19. The method of claim 18, wherein the creatinine pronutrient comprises creatine isopentyl ester hydrochloride or derivatives thereof.

20. The method of claim 18, wherein the creatinine pronutrient comprises creatine tert-butyl ester hydrochloride or derivatives thereof.
